(19) United States
(12) Patent Application Publication
(10) Pub. No.: US 2020/0001024 A1
(43) Pub. Date: Jan. 2, 2020

(54) LIQUID ATOMIZING DEVICE

(71) Applicant: YI-JIN PROMOLD ENTERPRISE LTD., Yunlin County (TW)

(72) Inventor: Yu-Chi CHEN, New Taipei City (TW)

(73) Assignee: YI-JIN PROMOLD ENTERPRISE LTD., Yunlin County (TW)

(21) Appl. No.: 16/037,032

(22) Filed: Jul. 17, 2018

(30) Foreign Application Priority Data
Jun. 28, 2018 (TW) 107122368

(51) Int. Cl. A61M 11/06; A61M 11/02; A61M 16/14; A61M 15/00

(52) U.S. Cl. CPC A61M 11/06; A61M 11/02; B05B 7/0433; A61M 15/0001; A61M 16/14

(57) ABSTRACT

A liquid atomizing device includes a medical solution cup connected to an air pump, and an output cover installed onto the medical solution cup and including an atomizing socket formed in the outer cover, and the top of the atomizing socket cover has a linear opening and a drainage groove for increasing the flow of a medical solution. When the medical solution is guided to flow upwardly through the drainage and sprayed from a specially designed linear opening to the outside through the drainage groove due to a high speed airflow, the medical solution is collided with a barrier rod disposed above the linear opening to form an atomized medical solution, and the linear opening has the effect of atomizing the medical solution stably.

### FIELD OF INVENTION

The present invention relates to a liquid atomizing device that contains a medical solution and is connected to an air pump, and the medical solution is atomized by the liquid atomizing device before being inhaled by a patient through the patient's nose or mouth.

### BACKGROUND OF INVENTION

### 1. Description of the Related Art

With reference to FIGS. 1 and 2 for a conventional liquid atomizing device, the liquid atomizing device comprises a medical solution cup 100 for containing a medical solution, and an output cover 200 installed onto the medical solution cup 100, wherein a conical jet pipe 101 is installed at the middle of the medical solution cup 100 and coupled to the air pump, and an external atomizing socket 201 is disposed at the bottom of the output cover 200 and sheathed on the conical jet pipe 101, and a gap 202 is formed between the outer periphery of the conical jet pipe 101 and the inner periphery of the atomizing socket 201 and communicated to the interior of the medical solution cup 100 for flowing the medical solution, so that the medical solution can be filled into the gap 202 according to a capillary action.

When a high speed airflow is entered into the conical jet pipe 101 and sprayed to the outside through a nozzle at the top, a pressure difference will be produced between the surrounding of the outlet at the top of the atomizing socket 201 and the conical jet pipe 101 (according to the Bernoulli's principle), so that the medical solution flows upwardly along the gap 202 between the outer periphery of the conical jet pipe 101 and the inner periphery of the atomizing socket 201, and then the medical solution is sprayed together with the high speed airflow to the outside from the top outlet of the atomizing socket 201 and collided with a baffle plate 203 to perform an atomization to produce an atomized medical solution which is provided to be inhaled through a patient's nose or mouth, so as to provide the atomized medical solution to the patient for treatment.

If the top outlet of the conventional atomizing socket 201 is a round hole 204, the output flow of the medical solution will be limited, and thus the generation rate of the atomized medical solution will also be limited. In the prior art as shown in the figures, a long-strip expansion slot 205 is formed separately on both sides of the round hole 204, and the two long-strip expansion slots 205 are extended downwardly from the top to the bottom of the atomizing socket 201 and directly communicated to the medical solution cup 100, and both sides of the two long-strip expansion slots 205 are communicated to the gap 202, so that the flow of the medical solution can be increased by the long-strip expansion slots 205.

Although the long-strip expansion slots 205 expand the passage for flowing the medical solution, the inventor of the present invention discovered that the long-strip expansion slots 205 affect or even ruin the capillary action occurred at the gap 202 between the outer periphery of the conical jet pipe 101 and the inner periphery of the atomizing socket 201 and reduce the area of the gap 202 and the content of the medical solution. In fact, the upward flow of the sprayed medical solution cannot be increased, but the airflow flows in an opposite direction from the long-strip expansion slots 205 into the gap 202 and the medical solution cup 100 to disturb the upward flow of the medical solution and fail to stably maintain the flow of the generated atomized medical solution.

In view of the aforementioned drawbacks of the prior art, the inventor of the present invention based on years of experience in the related industry to conduct extensive research and experiment, and finally designed a liquid atomizing device in accordance with the present invention to overcome the drawbacks of the prior art.

### 2. Summary of the Invention

It is a primary objective of the present invention to provide a liquid atomizing device to overcome the drawbacks of the prior art.

To achieve the aforementioned and other objectives, the present invention provides a liquid atomizing device, comprising: a medical solution cup coupled to an external air pump, and an output cover detachably installed onto the medical solution cup, and the medical solution cup having a medical solution storage tank, and the medical solution storage tank having a jet pipe protruded upwardly from the bottom of the medical solution storage tank, and the top of the jet pipe having a conical nozzle, and the output cover having an output flow passage formed therein and communicated to the outside, and an external atomizing socket disposed under the output flow passage and capable of sheathing the jet pipe, and a gap formed between the inner periphery of the atomizing socket and the outer periphery of the jet pipe for filling the medical solution stored in the medical solution storage tank into the gap, characterized in that:

The atomizing socket has a tapered portion disposed at the top inner periphery of the atomizing socket and superimposed on the top outer periphery of the conical nozzle to block the gap, and a linear opening formed at the top surface of the atomizing socket above the tapered portion for increasing the flow of the medical solution, and the linear opening includes two long sides and two short sides disposed between both ends of the two long sides.

A drainage groove is formed separately at the inner periphery of the two corresponding long sides, and the two drainage grooves are extended downwardly from the linear opening and communicated to the gap through the inner periphery of the tapered portion, so that the medical solution in the gap can be passed through the drainage groove and then sprayed to the outside.

A support is extended upwardly and separately from the outer sides of the two short sides and coupled to the output flow passages on both sides, and a parallel barrier rod is disposed above the linear opening and between the two supports for atomizing the medical solution after the medical solution is impacted.

The two short sides are extended downwardly to form an expansion slot with the outer periphery of the conical nozzle, and the two expansion slots are substantially tapered downward and the bottom of the expansion slot is near the outer periphery of the bottom of the conical nozzle for preventing the airflow in an opposite direction from flowing through the two expansion slots and injecting downwardly into the gap to disturb the medical solution that flows upwardly in the gap to the drainage groove.

In the design of the atomizing socket and the linear opening, the linear opening is provided for extending two edges of the upper surface of the tapered portion to expand the range of length as much as possible, so that when the high speed airflow is sprayed to the outside from the conical nozzle of the jet pipe, the medical solution in the medical solution storage tank is driven to spray from the linear opening to the outside through the drainage groove between the tapered portion and the conical nozzle, and the long-strip shaped and continuously opened structure of the linear opening improves the output flow of the medical solution, so that the sprayed medical solution can be collided with the barrier rod to produce an atomized medical solution, and the atomized medical solution is sent to the outside through the output flow passage of the output cover immediately, so as to improve the generation rate and stability of the atomized medical solution. This invention overcomes the drawback of the conventional liquid atomizing device that has an outlet in the shape of a round hole and limits the output flow of the medical solution, and prevents the generated atomized medical solution from failing to maintain its stability due to the long-strip expansion slots on both sides of the round-hole shaped outlet.

In an embodiment, the two expansion slots have a bottom near the outer periphery of the conical nozzle and maintaining a crevice smaller than the gap.

In an embodiment, the two expansion slots have a bottom abutting the outer periphery of the conical nozzle and isolated from the gap.

In an embodiment, the two drainage grooves of the top tapered portion of the atomizing socket are in a straight flat shape or an upwardly tapered shape.

In an embodiment, the barrier rod has a bump formed at the bottom of the barrier rod and facing the top of the draining groove and capable of bearing the impact of the medical solution.

In an embodiment, the output cover is integrally formed with the atomizing socket.

In an embodiment, the jet pipe has a bottom extended to an outer side of the medical solution cup for coupling the air pump.

### BRIEF DESCRIPTION OF THE DRAWINGS

FIG. 1 is a schematic view of a conventional liquid atomizing device;

FIG. 2 is a perspective view of a conventional liquid atomizing device;

FIG. 3 is a perspective view of a preferred embodiment of the present invention;

FIG. 4 is a cross-sectional view of FIG. 3;

FIG. 5 is a partial cross-sectional view of FIG. 3;

FIG. 6 is a bottom blowup view of the present invention;

FIG. 7 is a cross-sectional blowup view of the present invention;

FIG. 8 is another cross-sectional blowup view of the present invention;

FIG. 9 is a partial blowup view of FIG. 7;

FIG. 10 is a partial blowup view of FIG. 8;

FIG. 11 is a cross-sectional front view of FIG. 7;

FIG. 12 is a cross-sectional front view of FIG. 8;

FIG. 13 is a perspective view of an atomizing socket of the present invention;

FIG. 14 is a top view of FIG. 13; and

FIG. 15 is a perspective view of an additional embodiment as depicted in FIG. 13.

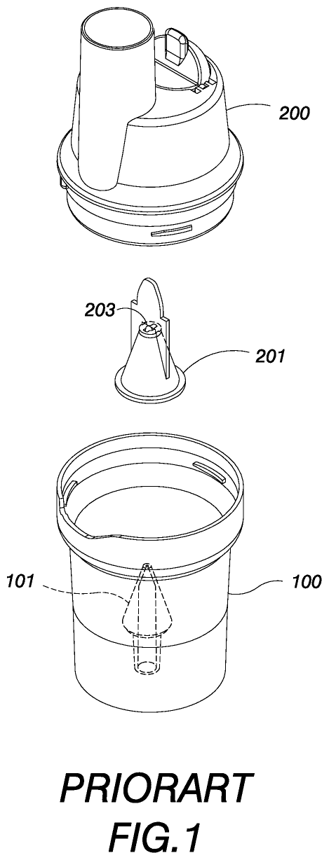

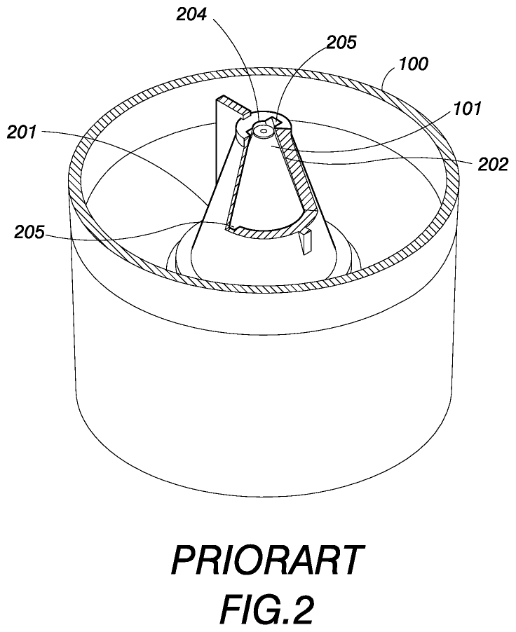

### DESCRIPTION OF THE PREFERRED EMBODIMENTS

To make it easier for our examiner to understand the objective, technical characteristics, structure, innovative features, and performance of the invention, we use preferred embodiments together with the attached drawings for the detailed description of the invention.

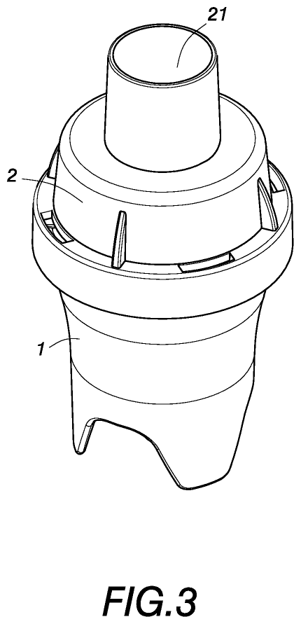

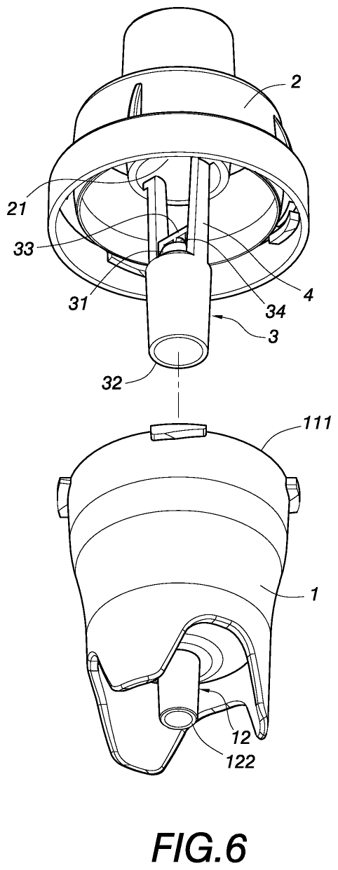

With reference to FIGS. 3 to 8 for a liquid atomizing device in accordance with a preferred embodiment of the present invention, the liquid atomizing device comprises: a medical solution cup 1 coupled to an external air pump, and an output cover 2 detachably installed onto the medical solution cup 1, and the medical solution cup 1 having a medical solution storage tank 11 disposed therein and opened upwardly and capable of containing a medical solution, a jet pipe 12 extended upwardly from the bottom of the medical solution storage tank 11, and the top 121 of the jet pipe 12 being disposed in the medical solution storage tank 11, a conical nozzle 13 disposed at the top 121 of the jet pipe 12, and the bottom 122 of the jet pipe 12 being extended to the outer side of the bottom of the medical solution cup 1 to connect an air pump (not shown in the figure).

In the figures, the output cover 2 is installed onto a tank opening 111 of the medical solution storage tank 11 of the medical solution cup 1, and the output cover 2 has an output flow passage 21 formed therein and communicated to the outside, and an atomizing socket 3 disposed under the output flow passage 21 and sheathed on the jet pipe 12 and the conical nozzle 13, wherein the output cover 2 and the atomizing socket 3 are integrally formed by in-mold injection molding.

The atomizing socket 3 has a top 31 and a bottom 32, and a gap t formed between the inner periphery 30 of the atomizing socket 3 (or an internal socket wall) and the outer periphery 120 of the jet pipe 12 (or an external socket wall) and upwardly communicated to the bottom of the medical solution storage tank 11, so that the medical solution can be filled into the gap t according to a capillary action.

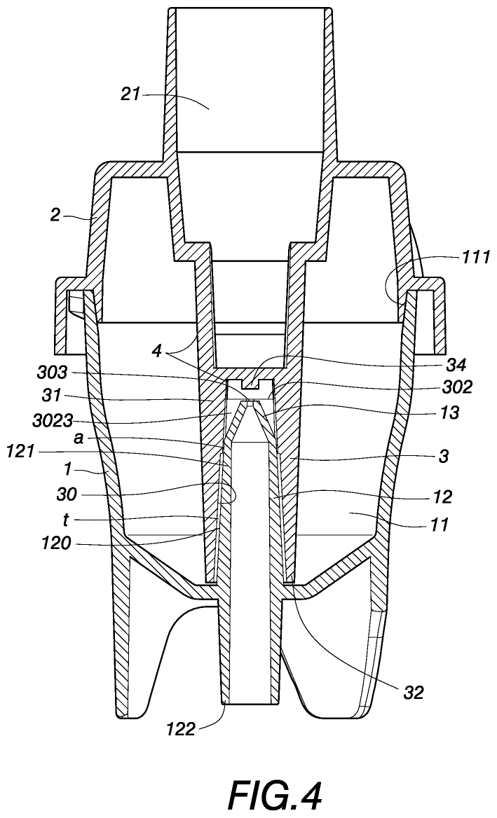

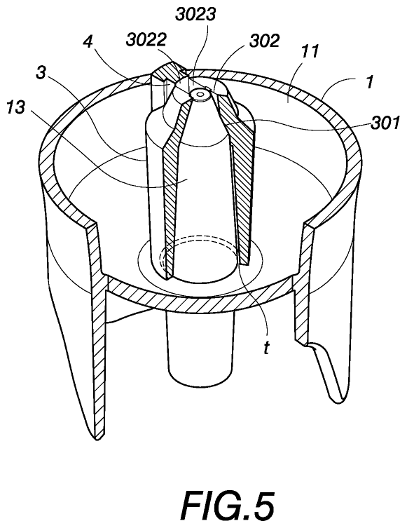

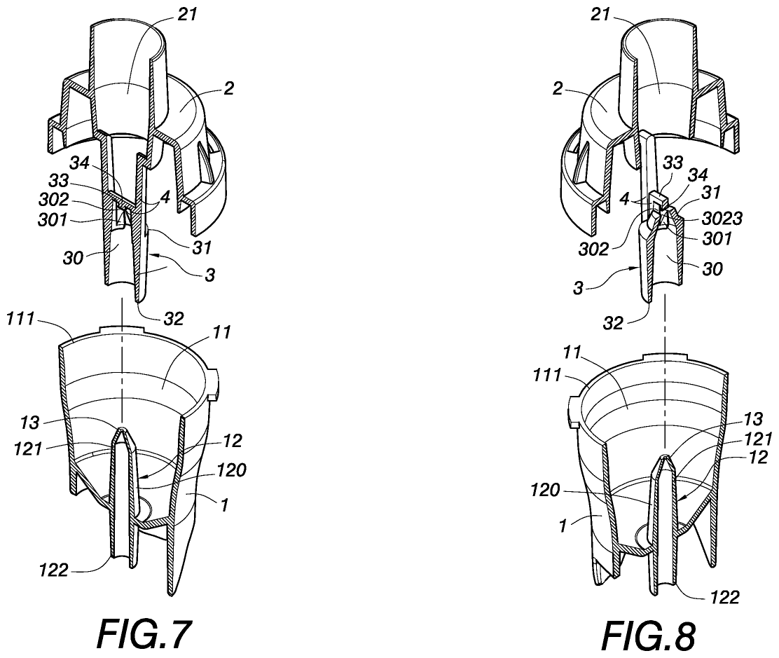

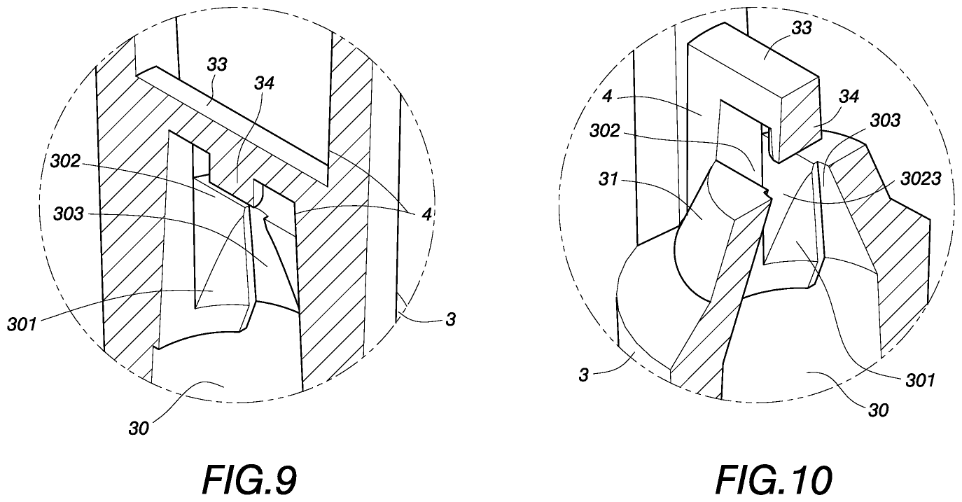

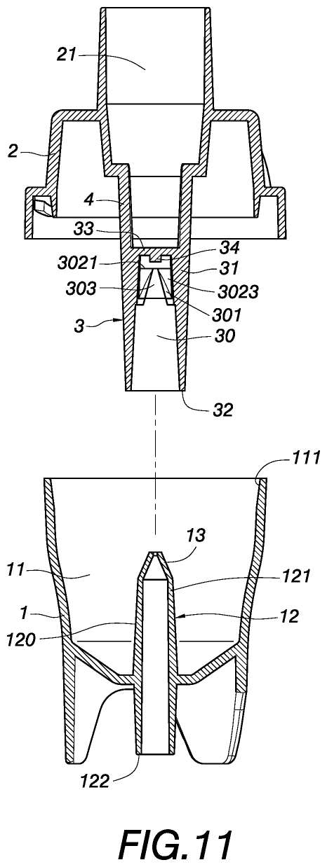

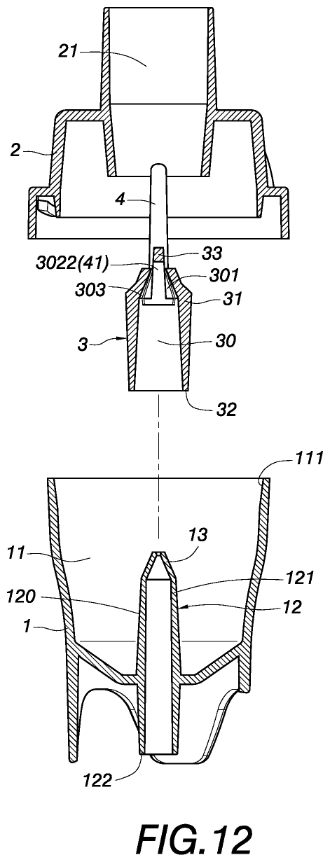

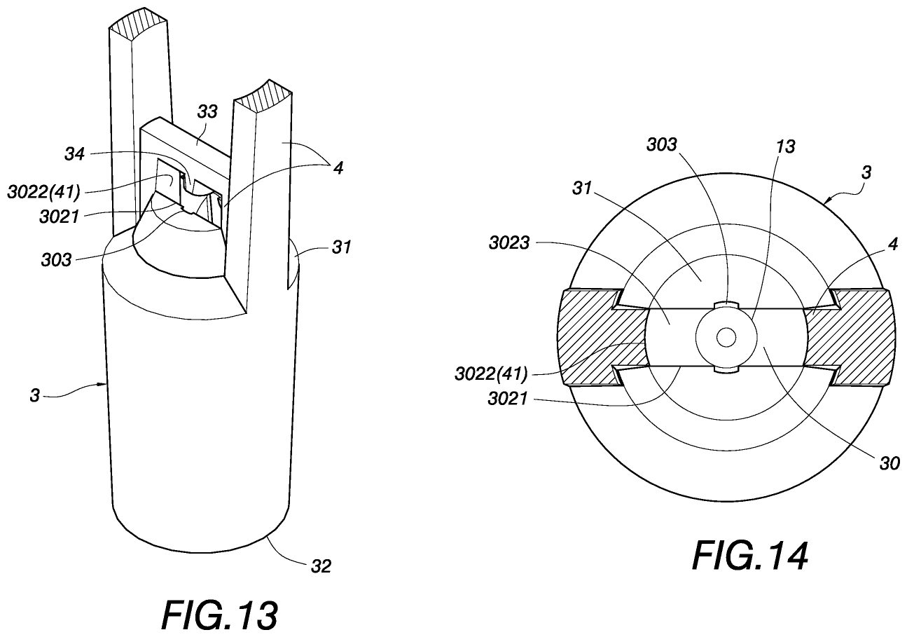

With reference to FIGS. 7 to 12 for a preferred embodiment of the present invention, a tapered portion 301 is disposed at the inner periphery 30 of the top 31 of the atomizing socket 3 and superimposed on the outer periphery of the top of the conical nozzle 13 to block the gap t (as shown in FIG. 4), wherein the top surface of the tapered portion 301 is a plane having a linear opening 302 capable of improving the flow of the gas and medical solution flow. In FIGS. 5, and 11 to 14, the linear opening 302 includes two long sides 3021 and two short sides 3022 disposed between both ends of the two long sides 3021.

Wherein, the corresponding two long sides 3021 of the linear opening 302 have a drainage groove 303 formed separately on both sides of the inner periphery 30 and extended downwardly from the long side 3021 of the linear opening 302, and communicated to the gap t through the inner periphery 30 of the tapered portion 301, wherein the two drainage grooves 303 of the tapered portion 301 at the top 31 of the atomizing socket 3 is substantially in a straight flat shape or an upwardly tapered shape, so that the medical solution in the gap t can be sprayed to the outside through the two drainage grooves 303.

A support 4 is extended vertically upward from the outer side of the two short sides 3022 of the linear opening 302 and coupled to both sides of the output flow passage 21, and a barrier rod 33 is disposed between the two supports 4 and above the linear opening 302, and extended parallel to the linear opening 302, and the bottom of the barrier rod 33 is provided to be collided for atomizing the medical solution.

In addition, the inner sides of the two short sides 3022 are extended downwardly to form an expansion slot 3023 with the outer periphery of the conical nozzle 13, and the two expansion slots 3023 are tapered downward and have a bottom proximate to the outer periphery of the conical nozzle 13 and maintain a crevice a (smaller than the gap t) with the gap t (as shown in FIG. 4); or, the two expansion slots 3023 have a bottom abutting the outer periphery of the conical nozzle 13 and separated from the gap t to prevent the airflow in an opposite direction from passing through the two expansion slots 3023 to disturb and hinder the medical solution in gap t from flowing upwardly to the drainage groove 303.

In addition, the inner side 41 of the two supports 4 is aligned evenly with the corresponding inner side of the two short sides 3022, so that the two long sides 3021 can be extended to both edges of the top surface of the tapered portion 301 to expand the linear opening 302 to a maximum length.

In a feasible embodiment, the barrier rod 33 has a bump 34 disposed at the bottom of the barrier rod 33 and facing the top of the drainage groove 303 for bearing the impact of the medical solution. The bump 34 increases the area of the bottom of the barrier rod 33 for bearing the impact of the medical solution in order to promote the atomization of the medical solution.

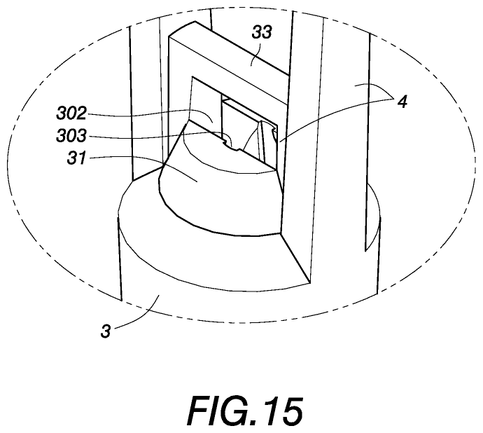

In FIG. 15, the impact of the medical solution may be withstood by the bottom of the barrier rod 33 alone.

In FIGS. 4 to 6, when a high speed airflow is sprayed from the conical nozzle 13 of the jet pipe 12 to the outside, the medical solution contained in the medical solution storage tank 11 is guided to flow upwardly along the gap t between the atomizing socket 3 and the jet pipe 12. The medical solution is passed through the drainage groove 303 between the tapered portion 301 and the conical nozzle 13 and then sprayed from the linear opening 302 to the outside. In addition, the long-strip shaped continuously opened structure of the linear opening 302 increases the area for passing and spraying the medical solution to the outside to improve the output flow of the medical solution, so that the sprayed medical solution can be collided with the barrier rod 33 and/or the bump 34 to produce an atomized medical solution, and the atomized medical solution is immediately passed through the output flow passage 21 of the output cover 2 to the outside, so as to improve the generation rate of the atomized medical solution and overcome the low speed of producing the atomized medical solution by the conventional liquid atomizing device.

In addition, the two expansion slots 3023 are formed between the inner sides of the two short sides 3022 of the linear opening 302 and the outer periphery of the conical nozzle 13 and tapered downward, and the bottom of the two expansion slots 3023 maintains a crevice a (smaller than the width of the gap) with the gap t, or the bottom of the two expansion slots 3023 is separated from the gap t to prevent the airflow in an opposite direction from filling upwardly from the bottom of the two expansion slots 3023 into the gap t to disturb and hinder the medical solution in the gap t from flowing upwardly to the drainage groove 303. The long-strip shaped continuously opened structure of the linear opening 302 increases the area for passing and spraying the atomized medical solution to the outside without affecting the content of the medical solution filled in the gap t while improving the stability of producing the atomized medical solution.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A liquid atomizing device, comprising a medical solution cup coupled to an external air pump, and an output cover detachably installed onto the medical solution cup, and the medical solution cup having a medical solution storage tank, and the medical solution storage tank having a jet pipe protruded upwardly from the tank bottom of the medical solution storage tank, and the top of the jet pipe having a conical nozzle, and the output cover having an output flow passage formed therein and communicated to the outside, and an external atomizing socket disposed under the output flow passage and capable of sheathing the jet pipe, and a gap formed between the inner periphery of the atomizing socket and the outer periphery of the jet pipe for filling the medical solution stored in the medical solution storage tank into the gap, characterized in that the top inner periphery of the atomizing socket has a tapered portion superimposed on the top outer periphery of the conical nozzle to block the gap, and a linear opening formed at the top surface of the atomizing socket above the tapered portion for increasing the flow of the medical solution, and the linear opening includes two long sides and two short sides disposed between both ends of the two long sides; a drainage groove is formed separately at the inner periphery of the two corresponding long sides, and the two drainage grooves are extended downwardly from the linear opening and communicated to the gap through the inner periphery of the tapered portion, so that the medical solution in the gap can be passed through the drainage groove and then sprayed to the outside; a support is extended upwardly and separately from the outer sides of the two short sides and coupled to the output flow passages on both sides, and a parallel barrier rod is disposed above the linear opening and between the two supports for atomizing the medical solution after the medical solution is impacted; and the two short sides are extended downwardly to form an expansion slot with the outer periphery of the conical nozzle, and the two expansion slots are substantially tapered downward and the bottom of the expansion slot is proximate to the outer periphery of the bottom of the conical nozzle for preventing the airflow in an opposite direction from flowing through the two expansion slots and injecting downwardly into the gap to disturb the medical solution that flows upwardly inside the gap to the drainage groove.

2. The liquid atomizing device of claim 1, wherein the two expansion slots have a bottom near the outer periphery of the conical nozzle and maintaining a crevice smaller than the gap.

3. The liquid atomizing device of claim 1, wherein the two expansion slots have a bottom abutting the outer periphery of the conical nozzle and isolated from the gap.

4. The liquid atomizing device of claim 1, wherein the two drainage grooves of the top tapered portion of the atomizing socket are in a straight flat shape or an upwardly tapered shape.

5. The liquid atomizing device of claim 1, wherein the barrier rod has a bump formed at the bottom of the barrier rod and facing the top of the draining groove and capable of bearing the impact of the medical solution.

6. The liquid atomizing device of claim 1, wherein the output cover is integrally formed with the atomizing socket.

7. The liquid atomizing device of claim 1, wherein the jet pipe has a bottom extended to an outer side of the medical solution cup for coupling the air pump.
